(19) United States
(12) Patent Application Publication
(10) Pub. No.: US 2004/0175332 A1
(43) Pub. Date: Sep. 9, 2004

(54) Dentrifice

(76) Inventor: Horst Westenfelder, Neustadt (DE)

Correspondence Address: Stephen M Haracz, Bryan Cave, 33rd Floor, 1290 Avenue of the Americas, New York, NY, 10104, US

(21) Appl. No.: 10/477,056

(22) PCT Filed: May 4, 2002
(86) PCT No.: PCT/EP02/04898

(30) Foreign Application Priority Data
May 10, 2001 (EP) 011114212

(51) Int. Cl. A61K 7/16; A61K 7/22

(52) U.S. Cl. 424/49; 424/54

(57) ABSTRACT

A method for suppressing discoloration of a composition in which a discoloration suppressing amount of ethylenediaminetetraacetic acid or a salt thereof is incorporated into a composition containing an ascorbic acid derivative. Compositions that resist discoloration containing an ascorbic acid derivative and ethylenediaminetetraacetic acid or a salt thereof, and methods of making such compositions.

The present invention is concerned with dentifrices. More particularly, the present invention is concerned with dentifrices containing ascorbic acid derivatives.

The term “dentifrice” as used herein denotes compositions for oral care comprising substantially solid or pasty formulations such as toothpaste, dental cream, gel or tooth powder.

Ascorbic acid derivatives, such as ascorbic acid phosphates, e.g., sodium ascorbyl phosphate is used in dentifrices to exert beneficial physiological effects, such as prevention of gingivitis or periodontitis. However, in dentifrices containing ascorbic acid derivatives such as ascorbic acid phosphate or sulphate esters and salts thereof discoloration has been observed on storage.

In accordance with the present invention it has been found that the discoloration of dentifrices containing ascorbic acid derivatives can be suppressed by the addition of ethylenediaminetetraacetic acid (EDTA) or salts thereof.

Thus, in one aspect, the present invention is concerned with the use of ethylenediaminetetraacetic acid or salts thereof for suppressing discoloration in dentifrices containing ascorbic acid derivatives.

Suitable salts of ethylenediaminetetraacetic acid are, e.g., alkali or earth alkali salts such as sodium, potassium, calcium or magnesium ethylenediaminetetraacetate. Mixtures of ethylenediaminetetraacetic acid and salts thereof may also be used. Preferred is ethylenediaminetetraacetic acid or sodium or calcium ethylenediaminetetraacetate.

The amount of ethylenediaminetetraacetic acid or a salt thereof is suitably from about 0.05 to about 5% by weight, preferably from about 0.1 to about 1% by weight of the total dentifrice composition, or from from about 0.01 to about 5% by weight, preferably from about 0.1 to about 2% by weight of the amount of ascorbic acid derivative present in the dentifrice composition. Typically, the amount of ascorbic acid derivative in the dentifrice is from about 0.01 to about 10% by weight, preferably from about 0.1 to about 5% by weight of the total dentifrice composition.

Examples of ascorbic acid derivatives present in the dentifrice are ascorbic acid esters, particularly phosphate esters and sulphate esters and salts thereof, or ascorbyl gluconate. Examples of salts are alkali and earth alkali metal salts, particularly sodium and magnesium salts. Of primary interest are sodium ascorbyl phosphate and magnesium ascorbyl phosphate.

In another aspect the present invention is concerned with dentifrices containing an ascorbic acid derivative as specified above and ethylenediaminetetraacetic acid or a salt thereof, except such dentifrices wherein the ascorbic acid derivative is magnesium ascorbyl phosphate and the ethylenediaminetetraacetic acid salt is calcium ethylenediaminetetraacetate.

Other components of the dentifrice of the present invention maybe those generally used in dentifrice compositions, e.g., abrasives such as silica, calcium carbonate, dicalcium phosphate, tricalcium phosphate, calcined alumina, sodium and potassium metaphosphate, sodium and potassium pyrophosphates, sodium trimetaphosphate, sodium hexametaphosphate and the like; thickening agents, such as guar gum, xanthan gum, caraya gum, carboxymethyl cellulose, hydroxymethyl cellulose, colloidal magnesium aluminum silicate and the like; surfactants such as hydrogenated coconut fatty acid monoglyceride monosulfate sodium, sodium lauryl sulfate, sodium dodecyl benzene sulfonate, N-lauroyl sarcosine, sorbitan monostearate and the like; fluorides and fluoride-providing compounds such as sodium, ammonium, calcium, zinc, copper and tin fluorides, sodium and ammonium fluorosilicate, sodium monofluorophosphate and the like; humectants such as glycerol, 1,4-butandiol, sorbitol, mannitol, xylitol and the like; metal salts such as cuprous fluoride, chloride and fluorosilicate, stannous fluoride and the like; flavoring agents such as peppermint, eucalyptus, cinnamon, lemon oil and the like; sweetening agents such as sucrose, xylitol, sodium cyclamate, saccharine and the like; antibacterial agents such as triclosan, chlorhexidine and the like; and various other adjuvants e.g. whitening or desensitizing agents. These ingredients may be incorporated in the dentifrice composition in amounts generally known to those skilled in the art.

The invention is illustrated further by the following Examples.

### EXAMPLE 1

A toothpaste can be prepared with the following ingredients:

| A) | Aqua | ad. 100 |
|---|---|---|
| | Glycerol | 30.00 |
| | Sorbitol | 20.00 |
| | EDTA | 0.01-1.00 |
| | Sodium Ascorbyl Phosphate | 0.01-5.0 |
| B) | Carboxy Methylcellulose | 1.2 |
| C) | Titanium Dioxide | 0.50 |
| | Sodium Cyclamate | 0.10 |
| | Sodium Saccharin | 0.01 |
| | Natrium mono-fluorophosphate | 0.85 |
| | Hydrated Silica | 20.00 |
| | Triclosan | |
| D) | Sodium Lauryl Sulfate | 1.00 |
| | Aroma | 0.80 |

### EXAMPLE 2

A transparent toothpaste can be prepared with the following ingredients:

| A) | Aqua | ad 100 |
|---|---|---|
| | Glycerol | 5.00 |
| | Sorbitol | 60.00 |
| | EDTA | 0.01-1.00 |
| | Sodium Ascorbyl Phosphate | 0.01-5.00 |
| B) | Carboxy Methylcellulose | 1.0 |
| C) | PEG-12 | 3.00 |
| | Saccharin | 0.20 |
| | Sodium Fluoride | 0.85 |
| | Hydrated Silica | 20.00 |
| D) | Sodium Lauryl Sulfate | 1.20 |
| | Aroma | 1.00 |

What is claimed is:

1. Use of ethylenediaminetetraacetic acid or a salt thereof for suppressing discoloration in dentifrices containing an ascorbic acid derivative.

2. The use as in claim 1 in a dentifrice containing an ascorbyl phosphate.

3. The use as in claim 2 wherein the ascorbyl phosphate is sodium ascorbyl phosphate.

4. The use as in any one of claims 1 to 3 in a dentifrice containing from about 0.01% by weight to about 5% by weight of an ascorbic acid derivative.

5. The use as in any one of claims 1 to 4 wherein the amount of ethylenediaminetetraacetic acid or a salt thereof is from about 0.01% by weight to about 5% by weight.

6. The use as in any one of claims 1 to 5 of ethylenediaminetetraacetic acid (EDTA).

7. The use as in any one of claims 1 to 5 of calcium ethylenediaminetetraacetate.

8. A dentifrice containing an ascorbic acid derivative and ethylenediaminetetraacetic acid or a salt thereof, except such dentifrices wherein the ascorbic acid derivative is magnesium ascorbyl phosphate and the ethylenediaminetetraacetic acid salt is calcium ethylenediarninetetraacetate.

9. The invention as particularly described hereinbefore, especially with reference to the Examples.
